(19) United States
(12) Patent Application Publication
(10) Pub. No.: US 2007/0166417 A1
(43) Pub. Date: Jul. 19, 2007

(54) Formulation and Methods for Use of Morinda Citrifolia Seed Oil

(76) Inventors: Afa K. Palu, American Fork, UT (US); Bing-Nan Zhou, Sandy, UT (US); Brett J. West, Orem, UT (US); Claude Jarake Jensen, Cedar Hills, UT (US); Stephen P. Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/561,987

(22) Filed: Nov. 21, 2006

Related U.S. Application Data
(60) Provisional application No. 60/740,446, filed on Nov. 29, 2005.

(51) Int. Cl. A61K 36/746; A61K 31/7048; A61K 31/353; A61K 31/202

(52) U.S. Cl. 424/776; 514/27; 514/560

(57) ABSTRACT

The present invention relates to nutraceutical formulations and methods for use of fetid oil derived from Morinda citrifolia seeds. Research supporting this invention indicates that processed Morinda citrifolia seed products may be used for weight management and health promotion. The present invention is supported by research findings related to the nutritional benefits of fetid oil extracted from the seeds contained within Morinda citrifolia fruit. This oil has been found to include high concentrations of linoleic acid, which is known to increase metabolic rate, help prevent cancer, protect the liver, and help prevent the build up of artery plaque. These observations are consistent with the concept that noni, can positively affect the regulation of excess body weight and promote overall good health.

### RELATED APPLICATIONS

This application claims priority to U.S. Provisional Application Ser. No. 60/740,446 filed Nov. 29, 2005, entitled “Nutraceutical Formulations and Methods for Use of Morinda Citrifolia Seed Oil”.

### BACKGROUND OF THE INVENTION

1. Field of the Invention

The present invention relates to formulations and methods for use of processed Morinda citrifolia products. Specifically, the present invention relates to nutraceutical formulations and methods for use of oil derived from Morinda citrifolia seeds.

2. Background and Related Art

The Indian Mulberry plant, known scientifically as Morinda citrifolia L., is a shrub, or small or medium sized tree 3 to 10 meters high. It grows in tropical coastal regions around the world. The plant grows randomly in the wild, and it has been cultivated in plantations and small individual growing plots. The Indian mulberry plant has somewhat rounded branches and evergreen, opposite (or spuriously alternate), dark, glossy, wavy, prominently-veined leaves. The leaves are broadly elliptic to oblong, pointed at both ends, 10-30 cm in length and 5-15 cm wide.

The Indian mulberry flowers are small, white, 3 to 5 lobed, tubular, fragrant, and about 1.25 cm long. The flowers develop into compound fruits composed of many small drupes fused into an ovoid, ellipsoid or roundish, lumpy body, 5-10 cm long, 5-7 cm thick, with waxy, white or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged, 2-celled stones, each containing 4 seeds.

When fully ripe, the fruit has a pronounced odor like rancid cheese. Although the fruit has been eaten by several nationalities as food, the most common use of the Indian mulberry plant was as a red and yellow dye source. Recently, there has been an interest in the nutritional and health benefits of the Indian mulberry plant.

It would be a significant advancement in the art to provide the essential oil from the Indian mulberry plant and to provide a process for obtaining the essential oil from the Indian mulberry plant.

### SUMMARY OF THE INVENTION

The present invention relates to formulations and methods for use of oil derived from Morinda citrifolia seeds. In some embodiments, processed Morinda citrifolia seed products may be used for weight management and general health promotion. Oil extracted Morinda citrifolia seeds by the methods disclosed include high concentrations of linoleic acid, and may be used to increase metabolic rate, help prevent cancer, protect the liver, and help prevent the build up of artery plaque. Accordingly, in some embodiments, various seed oil products can positively affect the regulation of excess body weight and promote overall good health.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

The foregoing and other features of the present invention will become more fully apparent from the accompanying drawings when considered in conjunction with the following description and appended claims. Although the drawings depict only typical embodiments of the invention and are thus, not to be deemed limiting of the invention's scope, the accompanying drawings help explain the invention in added detail.

FIG. 1 illustrates the effects of processing on noni seed oils phytoesterols;

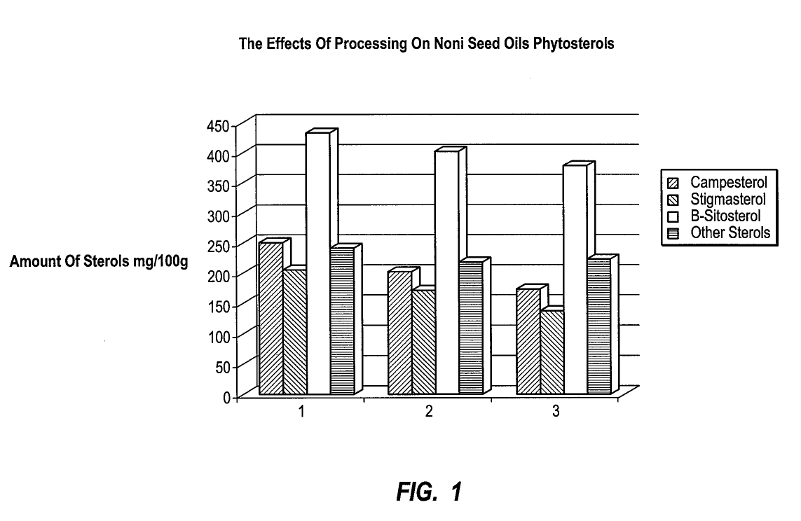

FIG. 2 illustrates effects of processing on beta-sitosterol in noni seed oil;

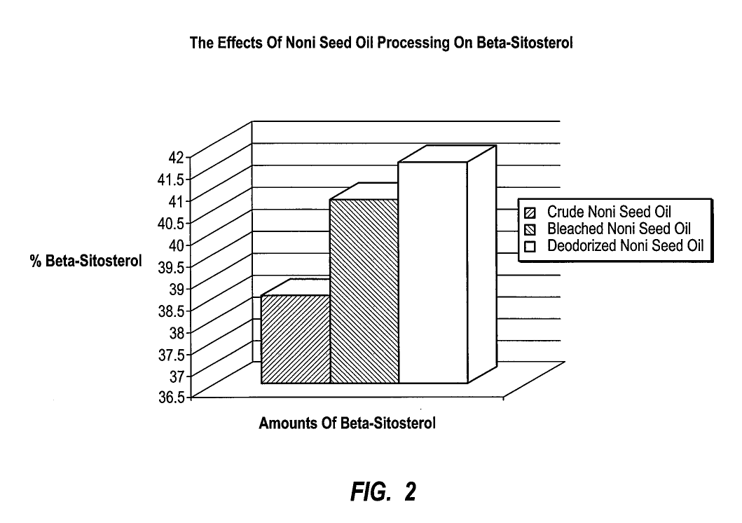

FIG. 3 illustrates the effects of processing on other sterols in noni seed oil;

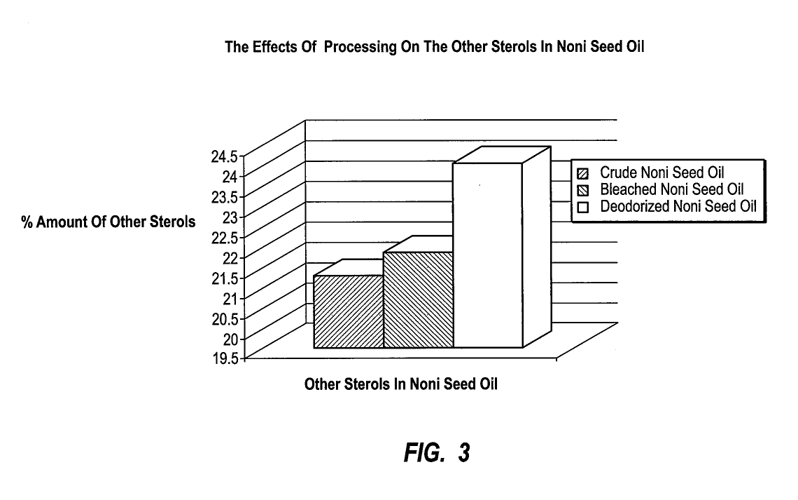

FIG. 4 illustrates the percentage of sterols in noni seed oils; and

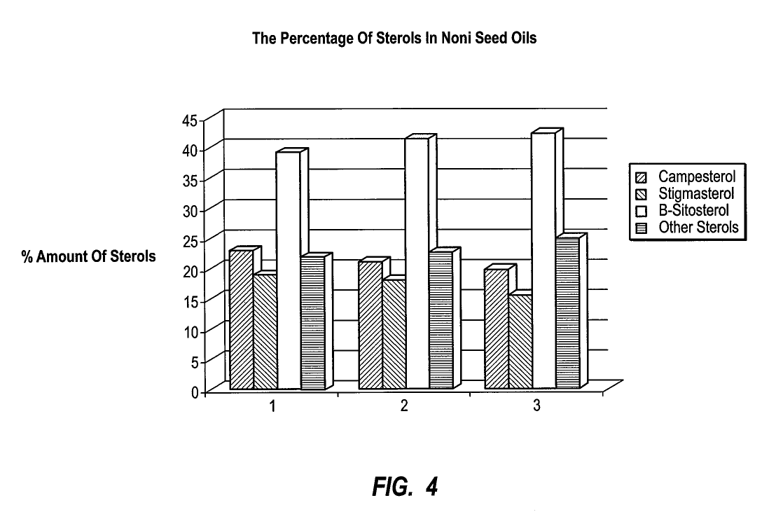

FIG. 5 illustrates the effects of processing on the vitamin E content of noni see oils.

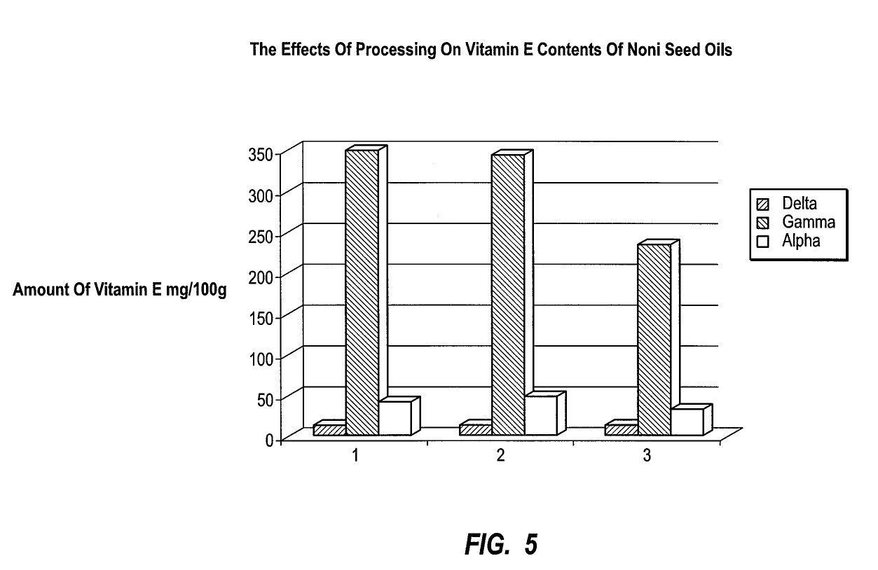

### DETAILED DESCRIPTION OF THE INVENTION

The present invention relates to formulations and methods for weight management, which utilize processed Morinda citrifolia products. The following disclosure of the present invention is grouped into subheadings, namely “General Discussion of Morinda citrifolia and the Methods Used to Produce Processed Morinda citrifolia Products” and “Formulations and Methods of Administration of Morinda citrifolia Products for Health Promotion including the use of Oil.” The utilization of the subheadings is for convenience of the reader only and is not to be construed as limiting in any sense.

### 1. General Discussion of Morinda citrifolia and the Methods Used to Produce Processed Morinda citrifolia Products

Morinda citrifolia fruit may be processed. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients, which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method weight management with a Morinda citrifolia-based nutraceutical formulation without any significant tendency to cause side effects.

### 2. Morinda citrifolia-based Nutraceutical Formulations and Methods of Administration for Health Promotion including the use of Fetid Oil

The present invention relates to formulations and methods for use of oil derived from Morinda citrifolia seeds. Research supporting this invention indicates that processed Morinda citrifolia seed products may be used for weight management and health promotion. The present invention is supported by research findings related to the nutritional benefits of oil extracted from the seeds contained within Morinda citrifolia fruit. Oil, secured by the methods disclosed herein, has been found to include high concentrations of linoleic acid, and may be used to increase metabolic rate, help prevent cancer, protect the liver, and help prevent the build up of artery plaque. Accordingly, in some embodiments, various products derived from the Morinda citrifolia plant, can positively affect the regulation of excess body weight and promote overall good health.

Some formulations disclosed herein include a high concentration of linoleic acid. Linoleic acid is a polyunsaturated fatty acid used in the biosynthesis of prostaglandins and cell membranes. Linoleic acid is particularly an Omega-6 essential fatty acid, which has been shown to promote numerous health benefits. Essential fatty acids (EFAs) are essential to human health but cannot be made in the body independently; for this reason, they must be obtained from food such as oil extracted from Morinda citrifolia seeds. Lineoleic acid is digested and converted to into Gamma-linoleic acid (GLA), a reaction catalyzed by the enzyme delta-6-desaturase (D6D). GLA is then broken down to arachidonic acid (AA) and/or another substance called dihomogamma-liolenic acid (DGLA).

Research has indicated that Gamma-linoleic acid (GLA) increases metabolism, which can effectively help reduce undesirable body weight. Likewise, evidence indicates that GLA can have positive anti-inflammatory properties by competing with arachidonic acid (AA). Other evidence indicates hepatoprotective (protection of liver function) and antiarterioscheotic (prevention of arterial plaque) properties of linoleic acid.

In one exemplary embodiment, the nutraceutical composition of the present invention comprises one or more of a processed Morinda citrifolia product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product, including fetid oil, may be prepared using a variety of techniques. According to one embodiment, the seeds from the Morinda citrifolia fruit are dried, preferably to a moisture content less than 10%. The seeds are ground or shredded to facilitate the removal of natural occurring oil. The shredded or ground flakes are pressed to expel Morinda citrifolia oil. The remaining seed cake is then mixed with a food grade, non-polar extraction solvent such as hexane. The mixture is heated for a sufficient length of time to complete the extraction process. The extraction solvent is then evaporated from the mixture leaving the Morinda citrifolia oil. The oil is further refined, bleached, dried, and deodorized to remove free fatty acids and other unwanted components. It should be noted that various other techniques may be used to produce a Morinda citrifolia product in accordance with desirable concentration characteristics including but not limited to linoleic acid concentration.

The processed Morinda citrifolia product is the active ingredient or contains one or more active ingredients, such as Quercetin and Rutin, and others, for effectuating natural control of the body weight of mammals. One embodiment of the present invention comprises a processed Morinda citrifolia product that promotes natural weight loss. Active ingredients may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The processed Morinda citrifolia product may be formulated with various other ingredients to produce various compositions, such as a nutraceutical composition, an internal composition, or others. The ingredients to be utilized in a nutraceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the nutraceutical composition will most likely be consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any known in the art.

In one exemplary embodiment, the present invention further features a method of administering a nutraceutical composition to a mammal for the purpose of weight management. The method comprises the steps of (a) formulating a nutraceutical composition comprising in part a processed Morinda citrifolia product present in an amount between about 0.01 and 95 percent by weight, wherein the composition also comprises a carrier, such as water or purified water, and other natural or artificial ingredients; (b) administering the nutraceutical composition into the body such that the processed Morinda citrifolia product is sufficiently internalized; (c) repeating the above steps as often as necessary to provide an effective amount of the processed Morinda citrifolia product.

The step of administering the nutraceutical composition into the body comprises ingesting the composition orally through one of several means. Specifically, the nutraceutical composition may be formulated as a liquid, gel, solid, or some other type that would allow the composition to be quickly and/or conveniently digested. Once sufficiently internalized, the administered nutraceutical composition may then begin to act to manage the weight of the subject. The management of weight may include administration of the nutraceutical composition to promote natural weight loss. The management of weight may include administration of the nutraceutical composition to maintain a desired body weight. Generally, it is contemplated that a broad range of objectives regarding the management of weight accomplished by consumption of products disclosed in the present invention may be accomplished by varying the formulation and administration procedures followed. In addition, the step of administering the nutraceutical composition may include injecting the composition into the body using an intravenous pump.

In one exemplary embodiment, the nutraceutical composition is administered by taking between 1 teaspoon and 2 oz., and preferably 2 oz., of the nutraceutical composition every two hours each day, or at least twice a day. The nutraceutical composition is to be taken on an empty stomach, meaning at a period of time at least two hours prior to consumption of any food or drink. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

Formulation One

| Ingredients | Percent by Weight |
|---|---|
| Morinda citrifolia puree juice or fruit juice | 100% |

Formulation Two

| Ingredients | Percent by Weight |
|---|---|
| Morinda citrifolia fruit juice | 50-99.99% |
| water | 0.1-50% |

Formulation Three

| Ingredients | Percent by Weight |
|---|---|
| Morinda citrifolia fruit juice | 50-99.99% |
| non-Morinda citrifolia-based fruit juices | 0.1-50% |

Formulation Four

| Ingredients | Percent by Weight |
|---|---|
| Morinda citrifolia fruit juice | 50-90% |
| water | 0.1-50% |
| non-Morinda citrifolia-based fruit juices | 0.1-30% |

Formulation Five

| Ingredients | Percent by Weight |
|---|---|
| Morinda citrifolia puree juice | 50-99.9% |
| water | 0.1-50% |

Formulation Six

| Ingredients | Percent by Weight |
|---|---|
| Morinda citrifolia puree juice | 50-99.9% |
| non-Morinda citrifolia-based fruit juices | 0.1-50% |

Formulation Seven

| Ingredients | Percent by Weight |
|---|---|
| Morinda citrifolia puree juice | 50-90% |
| water | 0.1-50% |
| non-Morinda citrifolia-based fruit juices | 0.1-30% |

Formulation Eight

| Ingredients | Percent by Weight |
|---|---|
| Morinda citrifolia dietary fiber | 0.1-50% |
| water | 1-99.9% |
| non-Morinda citrifolia-based fruit juices | 1-99.9% |

Formulation Nine

| Ingredients | Percent by Weight |
|---|---|
| Morinda citrifolia oil | 0.1-50% |
| carrier medium | 70-99.9% |
| other ingredients | 1-95% |

Formulation Ten

| Ingredients | Percent by Weight |
|---|---|
| Morinda citrifolia product | 10-80% |
| carrier medium | 20-90% |

Formulation Eleven

| Ingredients | Percent by Weight |
|---|---|
| Morinda citrifolia product | 5-80% |
| carrier medium | 20-95% |

Formulation Twelve

| Ingredients | Percent by Weight |
|---|---|
| Morinda citrifolia oil or oil extract | 0.1-50% |
| carrier medium | 20-90% |

Formulation Thirteen

| Ingredients | Percent by Weight |
|---|---|
| Morinda citrifolia puree juice or fruit Juice | 0.1-80% |
| Morinda citrifolia oil | 0.1-50% |
| carrier medium | 20-90% |

Formulation Fourteen

| Ingredients | Percent by Weight |
|---|---|
| Morinda citrifolia puree juice concentrate or | 100% |
| fruit juice concentrate | |

Formulation Fifteen

| Ingredients | Percent by Weight |
|---|---|
| Morinda citrifolia fruit juice | 50-99.8% |
| Morinda citrifolia oil | 0.1-50% |
| Formulation seventeen other fruit juices | 0.1-50% |
| Morinda citrifolia fruit juice concentrate or | 50-99.99% |
| puree juice concentrate | |
| water | 0.1-50% |

In one preferred method, a person wanting to manage their weight as described above takes, or is administered, at least one (1) ounce of Formulation One in the morning on an empty stomach, and at least one (1) ounce at night on an empty stomach, just prior to retiring to bed. In one example, which is not meant to be limiting in any way, the beneficial Morinda Citrifolia is processed into Tahitian Noni® juice manufactured by Morinda, Incorporated of Orem, Utah.

As stated, in one exemplary embodiment, the present invention features a method for introducing an internal composition of formulation to a subject for the purpose of weight management. This method essentially comprises the introduction of an internal composition, by oral consumption or otherwise, to the subject for the purpose of weight loss. Several embodiments of the internal comprising various different ingredients are contemplated for use herein, with each embodiment comprising one or more forms of a processed Morinda citrifolia product as taught and explained herein and a carrier agent or medium.

In one exemplary embodiment, the internal composition comprises the ingredients of: a processed Morinda citrifolia product present in an amount by weight between about 10-80 percent; and a carrier medium present in an amount by weight between about 20-90 percent. In this embodiment, the processed Morinda citrifolia product may comprise one or more of processed Morinda citrifolia fruit juice, processed Morinda citrifolia puree juice, processed Morinda citrifolia dietary fiber, and/or processed Morinda citrifolia oil extract.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a Morinda citrifolia dietary fiber product in similar concentrations.

In another exemplary embodiment Morinda citrifolia is administered at 0.25 ml/kg, 1 ml/kg or 4 ml/kg, for a series of days to accomplish the desired weight control.

According to the present invention, these particular methods of introducing an internal composition may comprise any method of actually introducing the internal composition to the subject for the purpose of weight management. Although the particular methods are many, the present invention recognizes that the internal composition may be introduced intravenously, transdermally, orally, or systemically. No matter what method is employed, it is important to regulate the amount of active ingredient that the subject is exposed to so that the appropriate weight management objectives are accomplished.

The carrier medium may comprise any ingredient capable of being introduced into the body of a mammal, and that is also capable of providing the carrying medium to the processed Morinda citrifolia product. Specific carrier mediums formulations are well known in the art and are not described in detail herein. The purpose of the carrier medium is as stated, to provide a means to embody the processed Morinda citrifolia product within the internal composition that is capable of being introduced into the body of the subject to be treated.

The following examples set forth and present the effects of Morinda citrifolia on the management of weight. These examples are not intended to be limiting in any way, but are merely illustrative of the benefits and advantages of utilizing Morinda citrifolia to regulate body weight.

### EXAMPLE 1

### Increased Metabolic Rate and Body Fat Reduction

The present invention relates to nutraceutical formulations and methods for weight regulation utilizing processed Morinda citrifolia products. One embodiment of the present invention comprises the oral administration of Morinda citrifolia products including fetid oil derived from Morinda citrifolia seeds, which increases the patient's metabolic rate and causes a reduction in body fat.

The properties of fetid oil from Morinda Citrifolia (noni) were analyzed by identifying the chemical composition of the oil in relation to known properties of the identified ingredients. The Morinda Citrifolia fetid oil was found to include a high concentration of linoleic acid. This acid is known as an essential fatty acid (EFA) because of its numerous health benefits and because of the human body's inability to produce the acid independently. One of the known properties of linoleic oil is the ability to positively affect metabolic rate. Therefore, formulations of the present invention will cause an increase in metabolic rate and a decrease in body fat in a manner known to be consistent with the ingestion of linoleic acid. In addition to metabolic rate, evidence indicates that linoleic acid helps prevent cancer, improve liver function, and minimize arterial plaque formation.

### EXAMPLE 2

### Regulation of Mammalian Body Weight

The present invention contemplates the use of nutraceutical formulations and methods for regulating mammalian body weight. The present invention contemplates the fact that some individuals will be interested in losing large amounts of weight while others will merely be interested in maintaining their body weight. The present invention contemplates a range of nutraceutical formulations and methods that may accommodate the varying weight regulation interests of specific individuals. The present invention contemplates utilizing variation in ingredients and dosage regimes to accomplish significant or minimal weight loss depending on the needs of the individual.

In an exemplary embodiment of the present invention, individuals could actualize weight loss from 0% of their body weight to nearly 50% of their body weight. This embodiment is supported by research conducted recently. Research performed supports the proposition that certain processed Morinda citrifolia products have a significant impact on weight loss.

### EXAMPLE 3

### Comedogenicity

Various Morinda citrfiolia seed oil products as disclosed herein maybe used typically without an increase in the number of acne comedones. A non-blinded, non-controlled prospective home use study of duration of four weeks was conducted. Twenty six (26) healthy teenage subjects were enrolled and twenty three (23) completed the study. No adverse reactions were reported.

Under the conditions employed in this study, no evidence of comedogenicity on the part of the test product was observed.

Twenty six healthy subjects were enrolled in the study, and twenty three completed the protocol. Subjects completed medical histories and informed consents prior to acceptance into the study.

A test area two by two centimeters in size was defined on the left side of each subject's forehead. Areas were examined visually under 15-2× magnification for the purpose of counting open comedones, Additionally, a modified pore biopsy was done on the application area for the purpose of counting closed comedones.

Subjects were instructed to attempt to apply equal amounts of product to the identical area each time. After four weeks, subjects returned for evaluation, and all observation modalities were repeated. Table I below contains descriptive statistics of comedone counts for the test panel. Values for individual participants can be found in the table below.

| | | | Closed Comedone | |
|---|---|---|---|---|
| | Open Comedone Counts | | Keratin Plug Counts | |
| | Baseline | End of Study | Baseline | End of Study |
| Average | 3.6 | 2.43 | 4.36 | 3.04 |
| Median | 2 | 1 | 4 | 3 |
| Standard | 6.96 | 3.68 | 3.23 | 2.18 |
| Deviation | | | | |

As can be seen in the table, the means and medians for both open and closed comedone counts decreased over the course of the study.

A two sample t-test assuming unequal variances was conducted on the percentage change values between baseline and the average of all subsequent ones to evaluate the hypothesis that mean of end-of-treatment comedone counts was not different than the baseline. Alpha was set at 0.05. By this test, the differences in the means appeared not to be significant for open or for closed comedones. Under the conditions employed in this study, no evidence of comedogenieity on the part of the test product was observed.

| | Open Comedone Counts | | Keratin Plug Counts | |
|---|---|---|---|---|
| Participant # | Baseline | End of Study | Baseline | End of Study |
| 129 | 2 | 1 | 3 | 3 |
| 152 | 2 | 2 | 6 | 2 |
| 327 | 1 | 1 | 3 | 2 |
| 435 | 3 | 2 | 4 | 3 |
| 437 | 5 | 2 | 4 | 6 |
| 421 | 2 | 1 | 1 | 1 |
| 420 | 1 | 1 | 1 | 1 |
| 326 | 2 | 1 | 3 | 2 |
| 623 | 4 | 4 | 6 | 3 |
| 375 | 1 | 1 | 1 | 1 |
| 376 | 1 | 1 | 5 | 3 |
| 436 | 1 | 1 | 6 | 4 |
| 449 | 1 | 1 | 6 | 2 |
| 448 | 2 | 1 | 3 | 1 |
| 372 | 1 | 1 | 2 | 1 |
| 602 | 3 | 3 | 2 | 4 |
| 503 | 2 | Dropped Test | 4 | Dropped Test |
| 294 | 20 | 14 | 8 | 5 |
| 295 | 20 | 10 | 14 | 10 |
| 267 | 1 | 1 | 2 | 3 |
| 268 | 2 | 1 | 2 | 2 |
| 505 | 2 | Dropped test | 2 | Dropped Test |
| 509 | 2 | 1 | 10 | 6 |
| 510 | 2 | 1 | 5 | 3 |
| 504 | 7 | 4 | 6 | 2 |
| Averages | 3.6 | 2.43 | 4.36 | 3.04 |
| Medians | 2 | 1 | 4 | 3 |
| Standard | 6.96 | 3.68 | 3.23 | 2.18 |
| Deviation | | | | |
| Variance | 26.67 | 12.69 | 6.57 | 3.88 |

### EXAMPLE 4

### Phytosterols

As a food additive, phytosterols have cholesterol-lowering properties (reducing cholesterol absorption in intestines), and may act in cancer prevention. Phytosterols naturally occur in small amount in vegetable oils, especially soybean oil. One such phytosterol complex, isolated from vegetable oil, is cholestatin, composed of campesterol, stigmasterol, and brassicasterol, and is marketed as a dietary supplement. Sterols can reduce cholesterol in human subjects by up to 15%.

β-sitosterol is one of several phytosterols with chemical structures similar to that of cholesterol. It is white in colour and waxy in nature. It is widely distributed in the plant kingdom and found in Serenoa repens (saw palmetto), Curcurbita pepo (pumpkin seed), Pygeum africanum, rice bran, wheat germ, corn oils, and soybeans.

Alone and in combination with similar phytosterols, β-sitosterol reduces blood levels of cholesterol. There is some belief that it plays some role in the possible benefits of herbal therapy of benign prostatic hypertrophy (BPH).

Phytosterols are present in Noni and processing affects the amount of phytosterols that are in the Noni seed oil. The biggest amount of sterols in the Noni seed oil is P-sitosterol and its presence is in decreasing amount with respect to the processing methods. Carnpeterol is next and behave like β-sitosterol. Stigmasterol is the next and behaves like the previous ones described earlier. The other sterols also behave like the other phytosterols that I previously described. It appears that the amount of phytosterols present in the Noni seed oils are decreasing and are affected by the methods of processing.

### Amounts of Beta-sitosterol

The amount of Beta-sitosterols present in the 3 different types of processing of the Noni seed oils as discussed below. It seems that the amount of beta-sitosterol is highest in the deodorized noni seed oil compared to the other processing.

The percentage amount of the other phytosterols mirrors those amounts for beta-sitosterols. It appears that the processing does affect the total percentage amounts of the other sterols in the Noni seed oils. Deodorized Noni seeds oil increases the amount of beta-sitosterol and also increases the amount of other sterols as well.

Campesterol and stigmasterol are decreasing while other sterols and beta-sitosterol are increasing as a result of different processing.

Various embodiments of products produces according to the invention comprise different ratios of vitamin E and beta_osterol. For example, if we want to have use a Noni seed oil that has the highest amount of vitamin E and highest amount of beta-sitosterol then one may use bleached Noni seed oil.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A formulation for regulating mammalian body weight, said formulation comprising a processed or extracted seed oil product from Morinda citrifolia.

2. A formulation of claim 1, wherein the extracted seed oil is produced by a method: wherein the method comprises: collecting Morinda citrfiolia fruit, allowing said fruit to dry to a moisture content of less than 10%, removing the seeds from the fruit, mechanically separating oil from said seeds, mixing the remaining seed cake with a solvent, heating the mixture, allowing the solvent to evaporate and leaving an oil product.

3. The formulation of claim 1, wherein the formulation is utilized for weight loss.

4. The formulation of claim 1, wherein the formulation is utilized for weight maintenance.

5. The formulation of claim 1, further comprising an item selected from al ist consisting of processed Morinda, Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, Morinda citrifolia brix.

6. The formulation of claim 1, further comprising an active ingredient selected from a list consisting of: Quercetin, rutin and lincoleic acid.

7. The formulation of claim 1, wherein said active ingredient is present in an amount between about 0.1 and 10% by weight.

8. The formulation of claim 1, further comprising juice from another fruit.

9. The formulation of claim 8, wherein the other fruit is selected from a list consisting of apple juice, blueberry juice and grape juice.

10. The formulation of claim 1, wherein said Morinda citrifolia product is used with a carrier medium.

11. The formulation of claim 1, wherein said composition is administered by process comprising one or more of the following methods: orally, transdermally to said infected area, by injection into said infected area, intravenously, applying composition topically and/or administered systemically.

12. The formulation of claim 1, wherein said formulation is processed to affect a consumer in a way selected form a list consisting of: increasing metabolic rate, reducing body fat, for hepatoprotective properties, for antiarterioscherotic properties, for regulating mammalian body weight, increasing metabolic rate, decreases body fat, and for treating obesity in mammals.

13. A method for regulating mammalian body weight, said method comprising: collecting Morinda citrfiolia fruit;
allowing said fruit to dry to a moisture content of less than 10%;
removing the seeds from the fruit;
mechanically separating oil from said seeds;
mixing the remaining seed cake with a solvent;
heating the mixture;
allowing the solvent to evaporate and leaving an oil product;
introducing the extracted seed oil product from Morinda citrifolia to said mammal.

14. The method of claim 13, further comprising the step of increasing metabolic rate in said mammals.

15. The method of claim 13, further comprising the steps of decreasing body fat in said mammals.

16. The method of claim 13, further comprising the steps of regulating mammalian body weight; increasing metabolic rate; and decreases body fat.

17. The method of claim 13, further comprising the step of treating obesity in said mammal.

18. A method for isolating an active ingredient in a processed Morinda citrifolia product and using said active ingredient to regulate mammalian body weight, said method comprising the steps of:
adding a Morinda citrifolia product to an alcohol-based solution;
isolating and extracting an active ingredient of said processed Morinda citrifolia product from said solution to obtain a fraction; and
introducing said extracted active ingredient into or onto said mammal, wherein said extracted active ingredient regulates mammalian body weight.

19. The method of claim 18, further comprising the steps of: collecting Morinda citrfiolia fruit, allowing said fruit to dry to a moisture content of less than 10%, removing the seeds from the fruit, mechanically separating oil from said seeds, mixing the remaining seed cake with a solvent, heating the mixture, allowing the solvent to evaporate and leaving an oil product.

20. The method of claim 18, wherein said solvent is selected from a list consisting of: water, butanol, methanol, ethanol, ethenol and ethylacetate.

21. The method of claim 18, wherein said active ingredient is selected from a list consisting of: Lineoleic acid, Quercetin and Rutin.

22. The method of claim 21, wherein said active ingredient is present between about 0.1 and 10% by weight.

23. The method of claim 17, wherein the extracted product is combined with an item selected from a list consisting of: Morinda Citrifolia fruit juice, Morinda citrifolia puree, another fruit juice, and a carrier prior to administration.
